(19) United States
(12) Patent Application Publication
(10) Pub. No.: US 2006/0084951 A1
(43) Pub. Date: Apr. 20, 2006

(54) Low energy of excitation PDT compounds for treatment of ocular disease

(76) Inventors: Gregory L. Heacock, Auburn, WA (US); John Marshall, Farnborough (GB); Paula A. Mahoney, Woodinville, WA (US)

Correspondence Address: MCANDREWS HELD & MALLOY, LTD, 500 WEST MADISON STREET, SUITE 3400, CHICAGO, IL, 60661, US

(21) Appl. No.: 11/240,403

(22) Filed: Sep. 30, 2005

Related U.S. Application Data
(60) Provisional application No. 60/616,239, filed on Oct. 5, 2004.

(51) Int. Cl. A61B 18/18

(52) U.S. Cl. 606/4; 607/88

(57) ABSTRACT

A photodynamic therapy includes a particular combination of a photosensitizer and light source for a photodynamic therapy used to treat ocular diseases. The photosensitizer has a peak excitation level when exposed to light of a given wavelength where the wavelength of light is absorbed at a particular location of the eye to be treated. The light source has a wavelength within the excitation range of the photosensitizer and matched to the location of the diseased eye tissue. The light source also has an irradiance level and energy level that is effective for therapy but at levels below the maximum permissible exposure level.

### CROSS-REFERENCE TO RELATED APPLICATIONS

The present application claims the priority of provisional patent application Ser. No. 60/616,239 filed Oct. 5, 2004. That application is hereby incorporated herein by reference.

### STATEMENT REGARDING FEDERALLY SPONSORED RESEARCH OR DEVELOPMENT

N/A

### FIELD OF THE INVENTION

The present invention is directed to a treatment for ocular disease using photodynamic therapy and more particularly to a combination of a photosensitizer and a light source for generating light at a wavelength within the excitation range of the photosensitizer and having an irradiance level and energy level that is effective for therapy but at levels below the maximum permissible exposure level.

### BACKGROUND OF THE INVENTION

In the treatment of ocular disease using photodynamic therapy (PDT), a photo reactive compound (also known as a “photosensitizer”) is injected into the patient. The photosensitizer concentrates in the diseased cells. Light of an appropriate wavelength is directed to the diseased eye tissue activating the photosensitizer to treat the diseased eye tissue. Eye conditions that may be treated by photodynamic therapy include, for example, age related macular degeneration (AMD), glaucoma or diabetic retinopathy, wherein photosensitizer inhibits the formation or retards the progression of the disease which is indicated by sub-retinal fluid concentration or rapid uncontrolled vascular growth in the diseased tissue.

Standards have been developed for surgery in the eye, and among these standards is a “maximum permissible exposure level” (MPE) which is the maximum energy per unit time of exposure. See ANSI 2136.1. However, the energy level of a laser used to activate the photosensitizer for many known procedures exceeds the MPE. For example, the current laser settings for some known photosensitizers is 1.4 times the MPE.

In addition, certain photosensitizers respond to wavelengths that are absorbed within intervening tissue in the eye. For example, the photosensitizer for treatment of AMD may be excited by wavelengths that are absorbed by intervening tissue so that light does not reach the AMD site in the intensity desired for treatment.

### BRIEF SUMMARY OF THE INVENTION

In accordance with the present invention, the disadvantages of prior photodynamic therapy treatments for ocular diseases have been overcome. The invention includes the combination of a photosensitizer and a light source for generating light at a wavelength within the excitation range of the photosensitizer and having an irradiance and energy level that is effective for therapy but at levels below the maximum permissible exposure level.

More particularly, the treatment for ocular disease in accordance with one embodiment of the present invention utilizes a photosensitizer that is activated by light have a wavelength in the range of 606 nm-720 nm in combination with a light source that generates light having a wavelength in the range of 606 nm-720 nm, and an irradiance in the range of 0.25 mw/cm²-350 mw/cm².

In another embodiment of the present invention, a photosensitizer that is activated by light having a wavelength in the range of 606 nm-720 nm is used in combination with a light source that generates light having a wavelength in the range of 606 nm-720 nm and an energy level in the range of 2 J/cm²-50 J/cm².

In a further embodiment, the present invention utilizes a photosensitizer that is activated by light have a wavelength in the range of 606 nm-720 nm in combination with a light source that generates light having a wavelength in the range of 606 nm-720 nm, and irradiance in the range of 0.25 mw/cm²-350 mw/cm² and an energy level in the range of 2 J/cm²-50 J/cm².

In accordance with another embodiment of the present invention, the photosensitizer is Talaporfin Sodium.

In accordance with a further embodiment of the present invention, the light source includes either a laser or one or more light emitting diodes.

In accordance with a further embodiment of the present invention, the spot size of the light for treatment is within the range of 200 microns-6000 microns.

These and other advantages and novel features of the present invention, as well as details of an illustrated embodiment thereof, will be more fully understood from the following description and drawings.

### BRIEF DESCRIPTION OF THE SEVERAL VIEWS OF THE DRAWING

FIG. 1 is a cross sectional view of an eye showing the absorption and degree of penetration into the eye of certain wavelengths of light;

FIG. 2 is a perspective view of a system for treating the eye in accordance with the present invention;

FIG. 3 is a perspective view of an optical module of the system shown in FIG. 2; and

FIG. 4 is rear view of the optical module of FIG. 3 illustrating the contact lens of the optical module.

### DETAILED DESCRIPTION OF THE INVENTION

The present invention provides a significant improvement in the safety and efficacy of ocular photodynamic therapy treatments that utilize photosensitizers and an excitation light source. In particular, the present invention provides a combination of a photosensitizer and light source for generating light at a wavelength within the excitation range of the photosensitizer for a given eye therapy wherein the light has an irradiance level and energy level that is effective for therapy but below the maximum permissible exposure level. The wavelength of light that is used is tailored to the location of the diseased eye tissue to be treated to minimize or prevent the light from exciting any photosensitizer that may be present in the intervening tissue between the light source and the diseased tissue. In accordance with the present invention, the irradiance of the light and/or energy level of the light are reduced to levels below the MPE. More particularly, the invention is directed to a particular combination of photosensitizer and light source having a wavelength matched to the excitation range of the photosensitizer and the tissue of the eye being targeted for treatment wherein the irradiance and energy level are within ranges that are effective for therapy but are at levels below the MPE.

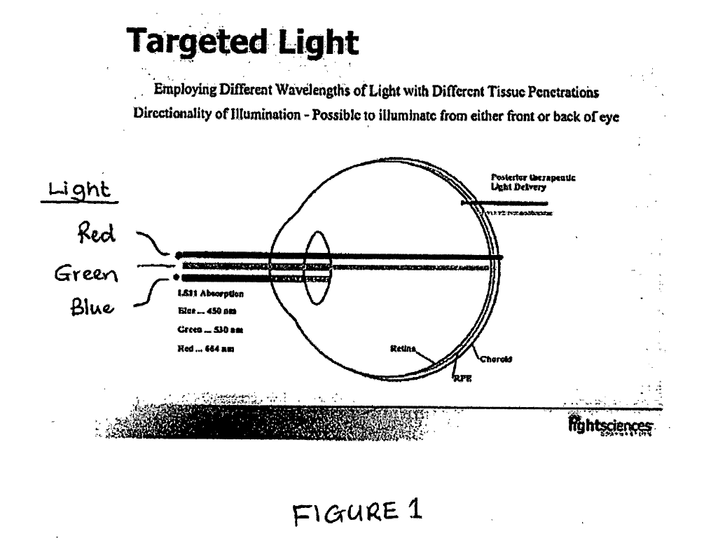

As shown in FIG. 1, different wavelengths of light are absorbed in eye tissues at different depths or locations within the eye. For example, light having a green wavelength is primarily absorbed in the retinal pigment epithelium; whereas light having a red wavelength is primarily absorbed in the layers of the choroid of the eye. In accordance with one embodiment of the present invention, the photosensitizer utilized is excited or is activated by light having a wavelength in the range of 606 nm-720 nm to treat diseased eye tissue in the back of the eye.

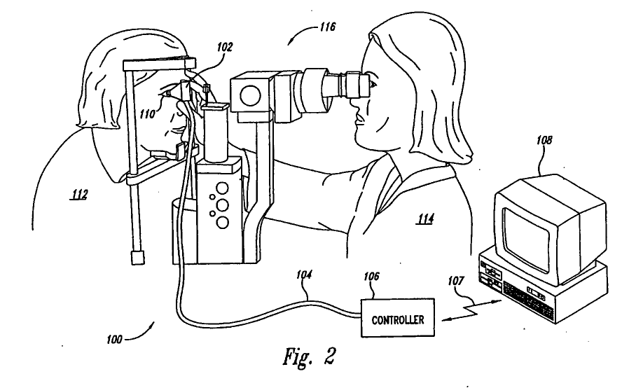

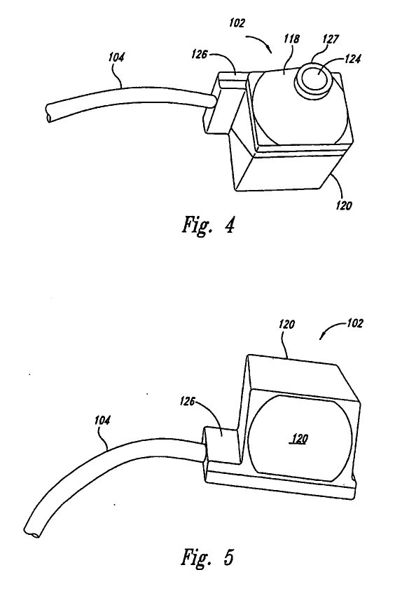

There are several known devices for projecting light into the eye for the purpose of photodynamic treatment. One example is a binocular microscope (slit lamp). However, the present invention can be adapted for use with virtually any device, as long as it is capable of generating or projecting light of the appropriate wavelength, irradiance level and energy level. FIGS. 2-4 illustrate an example of a suitable device. The device has an optical module 102 that includes a source of light which may be a laser. Alternatively, the light source may include one or more light emitting diodes (LEDs). The optical module also includes a known contact lens 110 for securing the position of the optical module 102 relative to the eye. An excitation system 100 includes the optical module 102 and a controller 106 coupled to the optical module by a cable 104. The controller 106 drives the light source and thus controls the irradiance of the light from the light source as well as the energy level of the light and the duration of the treatment, i.e. the duration that the photosensitizer in the diseased eye tissue is exposed to the light from the light source. The controller 106 may be coupled by a cable or by a wireless communication link 107 to a workstation computer 108 used in set-up and in the treatment. A typical treatment procedure involves the optical module 102 being placed on an eye 110 of a patient 112 by a clinician 114 to project light onto the diseased eye tissue after the photosensitizer has been injected into the patient. The clinician 114 typically views the eye 110 through a microspore 116 and a visualization interface 120 of the optical module 102 during treatment.

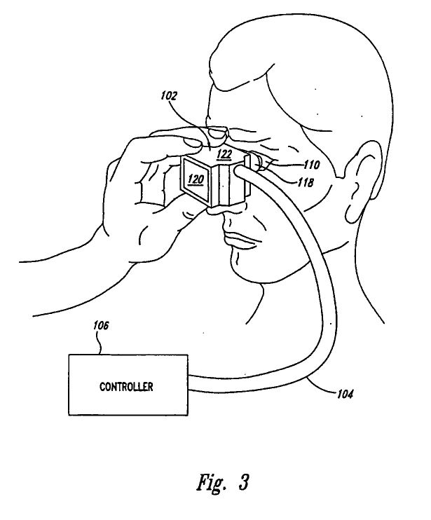

An implementation of the optical module 102 shown in FIGS. 3-5 includes a contact lens 124 to be placed on the eye 110 of the patient 112, a visualization interface 120 to allow a clinician to view the interior of the eye and a housing 122. The visualization interface 120 is typically either an optical lens or a viewing plane to allow the clinician 114 to view the eye 110 by placing the microscope 116 near to the visualization interface 120. The housing 122 contains the light source, which may be a laser or one or more LEDs, an optical lens or lenses and a reflector to reflect light from the light source to the eye and to pass light reflected from the eye to the visualization interface so that the clinician can view the interior of the eye. The contact lens 124 of the optical module 102 serves to neutralize the optical power of the cornea of the eye 110 so that treatment illumination may be directed to targeted eye tissue. The housing 122 may include a protrusion 126 or the like that can be gripped by a clinician to hold the contact lens on the eye 110. The housing also includes a mount 127 that is rotatably coupled to the contact lens 124 to allow for aiming and manipulation by the clinician 114.

In accordance with the present invention, the light generated by the light source of the optical module 102 has a wavelength in the range of 606 nm-720 nm; an irradiance in the range of 0.25 mw/cm²-350 mw/cm² and an energy level in the range of 2 J/cm²-50 J/cm² when used with a photosensitizer having excitation peaks at wavelengths in the range of 606 nm-720 nm. It is noted, for example that light having an irradiance of 300 mw/cm² which is used to excite the photosensitizer for a duration of approximately 2 minutes is well below the MPE. As the irradiance of light is decreased, the duration of exposure may increase and still be well below the MPE.

Suitable photosensitizers are described in U.S. Pat. No. 6,599,891, which is hereby incorporated by reference. In a preferred embodiment, the photosensitizer is Talaporfin Sodium. Talaporfin Sodium has a strong peak excitation level when exposed to light having a wavelength in the desired range, i.e. 606 nm-720 nm. Further, for AMD treatment, the preferred combination is a Talaporfin Sodium photosensitizer with light having an energy level of 2 J/cm²-50 J/cm²; an irradiance of 0.25 mw/cm²-350 mw/cm², and a therapy spot size 200-6000 microns.

Many modifications and variations of the present invention are possible in light of the above teachings. Thus, it is to be understood that, within the scope of the appended claims, the invention may be practiced otherwise than as described hereinabove.

What is claimed and desired to be secured by Letters Patent is:

1. A treatment for ocular disease comprising:
a photosensitizer that is activated by light having a wavelength in the range of 606 nm-720 nm; and
a light source for generating light having a wavelength in the range of 606 nm-720 nm, an irradiance in the range of 0.25 mw/cm²-350 mw/cm² and an energy level in the range of 2 J/cm²-50 J/cm².

2. A treatment for ocular disease as recited in claim 1 wherein the photosensitizer is Talaporfin Sodium.

3. A treatment for ocular disease as recited in claim 1 wherein the light source includes at least one light emitting diode.

4. A treatment for ocular disease as recited in claim 1 wherein the light source includes a laser.

5. A treatment for ocular disease as recited in claim 1 wherein the spot size of the light for treatment is within the range of 200 microns-6000 microns.

6. A treatment for ocular disease comprising:
a photosensitizer that is activated by light having a wavelength in the range of 606 nm-720 nm; and
a light source for generating light having a wavelength in the range of 606 nm-720 nm and an irradiance in the range of 0.25 mw/cm²-350 mw/cm².

7. A treatment for ocular disease as recited in claim 6 wherein the photosensitizer is Talaporfin Sodium.

8. A treatment for ocular disease as recited in claim 6 wherein the light source includes at least one light emitting diode.

9. A treatment for ocular disease as recited in claim 6 wherein the light source includes a laser.

10. A treatment for ocular disease as recited in claim 6 wherein the spot size of the light for treatment is within the range of 200 microns-6000 microns.

11. A treatment for ocular disease comprising:
a photosensitizer that is activated by light having a wavelength in the range of 606 nm-720 nm; and
a light source for generating light having a wavelength in the range of 606 nm-720 nm and an energy level in the range of 2 J/cm²-50 J/cm².

12. A treatment for ocular disease as recited in claim 11 wherein the photosensitizer is Talaporfin Sodium.

13. A treatment for ocular disease as recited in claim 11 wherein the light source includes at least one light emitting diode.

14. A treatment for ocular disease as recited in claim 11 wherein the light source includes a laser.

15. A treatment for ocular disease as recited in claim 11 wherein the spot size of the light for treatment is within the range of 200 microns-6000 microns.

16. A treatment for ocular disease comprising:
a photosensitizer that is activated by light having a wavelength in a predetermined excitation range for an eye therapy;
a light source for generating light in the excitation range of the photosensitizer for the eye therapy, the light having an irradiance in the range of 0.25 mw/cm²-350 mw/cm² and an energy level in the range of 2 J/cm²-50 J/cm².

17. A treatment for ocular disease as recited in claim 16 wherein the photosensitizer is Talaporfin Sodium.

18. A treatment for ocular disease as recited in claim 16 wherein the light source includes at least one light emitting diode.

19. A treatment for ocular disease as recited in claim 16 wherein the light source includes a laser.

20. A treatment for ocular disease as recited in claim 16 wherein the spot size of the light for treatment is within the range of 200 microns-6000 microns.

21. A treatment for ocular disease comprising:
a Talaporfin Sodium photosensitizer that is activated by light having a wavelength in the range of 606 nm-720 nm; and
a light source including at least one light emitting diode, the light source generating light having a wavelength in the range of 606 nm-720 nm, an irradiance in the range of 0.25 mw/cm²-350 mw/cm² and an energy level in the range of 2 J/cm²-50 J/cm².

22. A treatment for ocular disease as recited in claim 21 wherein the spot size of the light for treatment is within the range of 200 microns-6000 microns.

23. A treatment for ocular disease comprising:
a Talaporfin Sodium photosensitizer that is activated by light having a wavelength in the range of 606 nm-720 nm; and
a light source including laser, the light source generating light having a wavelength in the range of 606 nm-720 nm, an irradiance in the range of 0.25 mw/cm²-350 mw/cm² and an energy level in the range of 2 J/cm²-50 J/cm².
